(19) United States
(12) Patent Application Publication
(10) Pub. No.: US 2022/0175632 A1
(43) Pub. Date: Jun. 9, 2022

(54) OIL-IN-WATER EMULSION COSMETIC

(71) Applicant: Shiseido Company, Ltd., Chuo-ku, Tokyo (JP)

(72) Inventors: Yumi NASU, Tokyo (JP); Makoto UYAMA, Tokyo (JP); Yuri MATSUDA, Tokyo (JP)

(73) Assignee: Shiseido Company, Ltd., Chuo-ku, Tokyo (JP)

(21) Appl. No.: 17/599,444

(22) PCT Filed: Mar. 27, 2020
(86) PCT No.: PCT/JP2020/014021

§ 371 Date: Sep. 28, 2021

(30) Foreign Application Priority Data
Mar. 29, 2019 (JP) 2019-067675

(51) Int. Cl. A61K 8/06; A61K 8/58; A61K 8/86; A61K 8/87; A61K 8/81; A61Q 19/00

(52) U.S. Cl. CPC A61K 8/062; A61K 8/585; A61K 8/86; A61K 2800/594; A61K 8/8147; A61Q 19/00; A61K 2800/10; A61K 8/87

(57) ABSTRACT

The present invention provides an oil-in-water emulsion cosmetic, particularly a skin care cosmetic such as a skin lotion or a milky lotion, that has a large water fraction and a small oil fraction, exhibits an excellent stability over time while having an optimal feeling of use on the skin, and can provide an elasticity to the skin. An oil-in-water emulsion cosmetic according to the present invention is characterized by comprising: (a) a carboxy-modified silicone; (b) a hydrophobized polyether urethane; and (c) a carboxyvinyl polymer.

### TECHNICAL FIELD

The present invention relates to oil-in-water emulsion cosmetics. More specifically, the present invention relates to an oil-in-water emulsion cosmetic having a high water content, such as lotions and milky lotions, which is capable of providing improved skin compatibility, a feel of use, etc., and imparting firmness to the skin by incorporating a carboxy modified silicone, hydrophobic modified polyether urethane, and a carboxyvinyl polymer.

### BACKGROUND ART

Lotions and milky lotions are cosmetics generally applied to the skin after washing and are also called as skin care cosmetics that have a cleansing effect and simultaneously a skin conditioning effect that keeps a moisture balance of the skin as well as skin moisturizing and softening effects of the skin. Such skin care cosmetics generally have a high ratio of an aqueous ingredient content to an oily ingredient content and are demanded to be cosmetics that have good spreadability, easiness in compatibility, and a non-oily but refreshing feel of use to the skin.

Patent Document 1 discloses a method for producing a fine emulsion cosmetic. The production method includes mixing an acid part (an anionic surfactant) consisting of specific carboxy modified silicones, higher alcohol, a nonionic surfactant, an oil phase, an organic amine and/or an alkali metal, a part of an aqueous phase, and dihydric alcohol to prepare a microemulsion, and subsequently adding the balance of the aqueous phase for dilution. When this method is used, a fine emulsion having an average emulsion particle size of 150 nm or less can be obtained without applying a high shear force by a high-pressure emulsifier. The internal phase (oil phase) of this fine emulsion forms an a gel including an oil content having a silicone oil or a hydrocarbon oil as the main component. As a result, when such a fine emulsion is applied to the skin, a refreshing feel without stickiness is imparted. However, this fine emulsion cosmetic failed to impart firmness to the skin.

Patent Document 2 discloses an elastic gel composition with improved viscosity stability at a high temperature. This elastic gel composition can be obtained by incorporating hydrophobic modified polyether urethane in an oil-in-water emulsion composition including fine oil drops (a gel) having an average particle size of 150 nm or less. This elastic gel composition containing the hydrophobic modified polyether urethane, that has a thickening effect, can impart firmness to the skin and also maintain a distinctive feel of use to a hydrophobic modified polyether urethane-containing composition (a jelly-like elastic unique feel when touched).

However, when hydrophobic modified polyether urethane is added to the fine emulsion of Patent Document 1, a problem is posed in that the emulsion stability at a high temperature declines. Further, it is described that the unique sense of touch provided by the composition of Patent Document 2 derives from the hydrophobic modified polyether urethane, but when other thickeners such as a carboxyvinyl polymer are further added, such a unique sense of touch is lost (Patent Document 2; Comparative Examples 9 and 10).

### CITATION LIST

### Patent Document

Patent Document 1: JP-B 6110450
Patent Document 2: JP-B 6113695

### SUMMARY OF INVENTION

### Technical Problem

An object of the present invention is to provide an oil-in-water emulsion cosmetic that has a high water content and a low oil content, specifically skin care cosmetics such as lotions and milky lotions having an optimal feel of use to the skin, excellent stability over time, and also capable of imparting firmness to the skin.

### Solution to Problem

As a result of extensive studies, the present inventors have found that, by incorporating a carboxyvinyl polymer in addition to hydrophobic modified polyether urethane in an oil-in-water emulsion cosmetic in which oil phases having an a gel structure including a carboxy modified silicone are dispersed, a cosmetic can be obtained that is capable of maintaining a sense of touch unique to the hydrophobic modified polyether urethane, having good stability over time and an optimal feel of use to the skin, and also imparting firmness to the skin, whereby the present invention has been accomplished.

That is, the present invention provides the oil-in-water emulsion cosmetic comprising:

(a) a carboxy modified silicone;
(b) hydrophobic modified polyether urethane; and
(c) a carboxyvinyl polymer.

### Advantageous Effects of Invention

The oil-in-water emulsion cosmetic of the present invention, which is an emulsion system containing a carboxy modified silicone, imparts a silky feel of use without stickiness. Additionally, the cosmetic has the unique jelly-like sense of touch derived from hydrophobic modified polyether urethane, good stability over time, and can impart firmness to the skin. That is, the cosmetic is particularly suitable for skin care cosmetics such as lotions and milky lotions.

In the oil-in-water emulsion cosmetic of the present invention, a carboxyvinyl polymer, which is considered to inhibit the unique sense of touch derived from the hydrophobic modified polyether urethane in Patent Document 2, is intentionally incorporated, whereby the unique usability derived from the hydrophobic modified polyether urethane can be maintained. In addition, the emulsion cosmetic also has excellent emulsion stability and imparts firmness to the skin. That is, the cosmetic of the present invention can be said to have the optimal feel of use to the skin as the skin care cosmetic that has a high water content and a low oil content. Further, the cosmetic of the present invention can be adjusted to have a flowable low viscosity and can thus be a cosmetic easily discharged from a dispenser with smooth usability.

### DESCRIPTION OF EMBODIMENTS

The oil-in-water emulsion cosmetic according to the present invention (simply referred also to as “the cosmetic”) contains (a) a carboxy modified silicone, (b) hydrophobic modified polyether urethane, and (c) a carboxyvinyl polymer as the essential ingredients. Details will be described below.

### (a) Carboxy Modified Silicone

The carboxy modified silicone (ingredient a) used in the present invention is a silicone modified with at least 1 carboxyalkyl group (an alkyl group having 2 to 22 carbon atoms). The average total number of silicon atom included in a molecular of such a carboxy modified silicone preferably ranges from 2 to 20, more preferably ranges from 3 to 18, and particularly preferably ranges from 3 to 7.

(a) The carboxy modified silicone in the present invention is preferably a compound represented by the following formula (1):

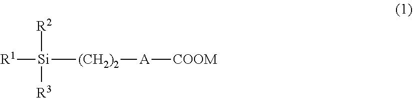

In the above formula (1), at least one of R¹ to R³ is a functional group represented by —O—Si(R⁴)₃ (R⁴ is an alkyl group or a phenyl group having 1 to 6 carbon atoms). All of R¹ to R³ can be the group corresponding to the above functional group (—O—Si(R⁴)₃). Alternatively, one or two of R¹ to R³ are the groups corresponding to the above functional group (—O—Si(R⁴)₃), and other(s) of R¹ to R³, which may be the same or different, can be substituted or unsubstituted monohydric hydrocarbon groups.

When R⁴ in the functional group represented by —O—Si(R⁴)₃ is an alkyl group having 1 to 6 carbon atoms, examples of such an alkyl group include linear, branched chain, and cyclic alkyl groups such as methyl, ethyl, n-propyl, i-propyl, n-butyl, butyl, s-butyl, t-butyl, pentyl, neopentyl, cyclopentyl, and hexyl. That is, specific examples of the functional group represented by —O—Si(R⁴)₃ include —O—Si(CH₃)₃, —O—Si(CH₃)₂(C₂H₅), —O—Si(CH₃)₂(C₃H₇), —O—Si(CH₃)₂(C₄H₉), —O—Si(CH₃)₂(C₅H₁₁), —O—Si(CH₃)₂(C₆H₁₃), and —O—Si(CH₃)₂(C₆H₅), but are not limited thereto. Of these, the functional group is preferably a trialkylsiloxy group, with a trimethylsiloxy group being most preferable.

Further, in the above formula (1), R¹ to R³ other than the above functional group (—O—Si(R⁴)₃) (“other R¹ to R³”), which may be the same or different, are substituted or unsubstituted monohydric hydrocarbon groups. Examples of the unsubstituted monohydric hydrocarbon group herein include linear, branched chain, and cyclic alkyl groups such as methyl, ethyl, n-propyl, i-propyl, n-butyl, i-butyl, s-butyl, t-butyl, pentyl, neopentyl, cyclopentyl, and hexyl; aryl groups such as a phenyl group, a tolyl group, and a xylyl group; and an aralkyl group. Examples of the substituted monohydric hydrocarbon group include perfluoro alkyl groups such as a 3,3,3-trifluoropropyl group and a 3,3,4,4,4-pentafluorobutyl group; aminoalkyl groups such as a 3-aminopropyl group and a 3-(aminoethyl)aminopropyl group; and amidoalkyl groups such as an acetylaminoalkyl group. Additionally, a part of the monohydric hydrocarbon group can further be substituted with a hydroxyl group, an alkoxy group, a polyether group, or a perfluoro polyether group, and examples of the alkoxy group include a methoxy group, an ethoxy group, and a propoxy group.

The “other R¹ to R³” is preferably a linear or branched chain alkyl group having 1 to 6 carbon atoms and, of these, a methyl group and an ethyl group are particularly preferable. That is, in the formula (1), all or two of R¹ to R³ are preferably the functional group represented by the —O—Si(R⁴)₃, and other R¹ to R³ are preferably a methyl group or an ethyl group.

M is a hydrogen atom, a metal atom, or an organic cation. Examples of the metal atom include a monohydric alkali metal, a dihydric alkaline earth metal, and other metal atoms. Examples of the monohydric alkali metal include Li, Na, and K, examples of the dihydric alkaline earth metal include Mg, Ca, and Ba, and others include Mn, Fe, Co, Al, Ni, Cu, V, Mo, Nb, Zn, and Ti. Examples of the organic cation include ammonium ion, monoethanol ammonium ion, triethanol ammonium ion, arginine neutralizing ion, and aminomethyl propanol (AMP) neutralizing ion. M is particularly preferably a hydrogen atom or a monohydric alkali metal or can be a mixture thereof.

A is a linear or branched chain alkylene group represented by C_{q}H_{2q}, and q is an integer of 0 to 20. In the present invention, q is preferably 2 to 15, and further preferably 6 to 12. On the other hand, when q is a value of more than 20, a feel of use when touched may be poor.

When q=0, the carboxy modified silicone represented by the formula (1) corresponds to a compound represented by the following formula (1′):

R¹R²R³Si—(CH₂)₂—COOM (1′)

This is the compound in which a carboxy group is bonded to a silicon atom through an ethylene group.

For the carboxy modified silicone represented by the above formula (1) in the present invention, a carboxy modified silicone wherein R¹ and R² are a functional group represented by —O—Si(R⁴)₃ (R⁴ is an alkyl group having 1 to 6 carbon atoms), R³ is a linear or branched chain alkyl group having 1 to 6 carbon atoms, and a value of q is 6 to 12 is particularly preferably used.

Particularly preferable examples include “carboxydecyl trisiloxane” (INCI name), that is, “3-(10-carboxydecyl)-1,1,1,3,5,5,5-heptamethyltrisiloxane”, and this compound is commercially available under the product name, “OP-1800MF Carboxy Fluid” (manufactured by Toray Dow Corning Corporation).

The amount of the carboxy modified silicone incorporated in the cosmetic of the present invention relative to the total amount of the cosmetic is 0.1 mass % or more, preferably 0.3 mass % or more, and more preferably 0.5 mass % or more. The upper limit of amount incorporated relative to the total amount of the cosmetic is 5 mass % or less, preferably 3 mass % or less, and more preferably 1 mass % or less. That is, the range of amount incorporated can be set, for example, from 0.1 to 5 mass %, 0.3 to 3 mass %, and 0.5 to 1 mass %, but is not limited thereto. However, when an amount of the carboxy modified silicone incorporated is less than 0.1 mass % or more than 5 mass %, a feel of use tends to decline.

### (b) Hydrophobic Modified Polyether Urethane

For the hydrophobic modified polyether urethane (ingredient b) incorporated in the cosmetic of the present invention, a compound represented by the following formula (2):

R⁵—{(O—R⁶)ₖ—OCONH—R⁷[—NHCOO—(R⁸—O)ₙ—R⁹]ₕ}ₘ (2)

is preferably used.

In the above formula (2), R⁵, R⁶ and R⁸ each independently represent a hydrocarbon group having 2 to 4 carbon atoms, preferably an alkyl group or an alkylene group having 2 to 4 carbon atoms.

R⁷ represents a hydrocarbon group having 1 to 10 carbon atoms and optionally having a urethane bond.

R⁹ represents a hydrocarbon group having 8 to 36, and preferably 12 to 24 carbon atoms.

m is the number of 2 or more, and preferably 2. h is the number of 1 or more, and preferably 1. k is the number of 1 to 500, and preferably 100 to 300. n is the number of 1 to 200, and preferably the number of 10 to 100.

The hydrophobic modified polyether urethane represented by the above formula (2) can be obtained by reacting, for example, one or two or more polyether polyols represented by R⁵—[(O—R⁶)ₖ—OH]ₘ (R⁵, R⁶, k, m herein are defined as above), one or two or more polyisocyanates represented by R⁷—(NCO)ₕ₊₁ (R⁷ and h herein are defined as above) and one or two or more polyether monoalcohols represented by HO—(R⁸—O)ₙ—R⁹ (R⁸, R⁹, and n herein are defined as above).

In this production method, R⁵ to R⁹ in the formula (2) are determined by R⁵—[(O—R⁶)ₖ—OH]ₘ, R⁷—(NCO)ₕ₊₁, and HO—(R⁸—O)ₙ—R⁹ that are to be raw materials. The ratio to be charged of the above three materials is not particularly limited but the ratio of the hydroxyl group derived from the polyether polyols and polyether monoalcohols to the isocyanate group derived from the polyisocyanates is preferably NCO/OH=0.8:1 to 1.4:1.

The polyether polyol compound represented by the above formula R⁵—[(O—R⁶)ₖ—OH]m can be produced by addition polymerization of an alkylene oxide, such as an ethylene oxide, a propylene oxide, a butylene oxide and an epichlorohydrin, or a styrene oxide to a m-hydric polyol.

For the polyols herein, 2 to 8 hydric polyols are preferable, and examples include dihydric alcohols such as an ethylene glycol, a propylene glycol, a butylene glycol, a hexamethylene glycol, and a neopentyl glycol; trihydric alcohols such as a glycerin, a trioxyisobutane, a 1,2,3-butanetriol, a 1,2,3-pentatriol, a 2-methyl-1,2,3-propanetriol, a 2-methyl-2,3,4-butanetriol, a 2-ethyl-1,2,3-butanetriol, a 2,3,4-pentatriol, a 2,3,4-hexanetriol, a 4-propyl-3,4,5-heptanetriol, a 2,4-dimethyl-2,3,4-pentatriol, a pentamethylglycerin, a pentaglycerin, a 1,2,4-butanetriol, a 1,2,4-pentatriol, a trimethylolethane, and a trimethylolpropane; tetrahydric alcohols such as a pentaerythritol, a 1,2,3,4-pentanetetrol, a 2,3,4,5-hexanetetrol, a 1,2,4,5-pentanetetrol, and a 1,3,4,5-hexanetetrol; pentahydric alcohols such as an adonit, an arabite, and a xylit; hexahydric alcohols such as a dipentaerythritol, a sorbit, a mannitol, and an iditol; and octahydric alcohols such as a sucrose.

R⁶ is determined by the alkylene oxide, styrene oxide or the like to be added, but alkylene oxides or styrene oxides having 2 to 4 carbon atoms are preferable due to particularly easy availability and to exhibit excellent effects. The alkylene oxide, styrene oxide or the like to be added can be homopolymerization or can be random polymerization or block polymerization of two or more thereof. The addition method can be a typical method. The polymerization degree k is 1 to 500. The proportion of the ethylene group in R⁶ is preferably 50 to 100 mass % of the entire R⁶. The molecular weight of R⁵—[(O—R⁶)ₖ—OH]m is preferably 500 to 100,000, and particularly preferably 1,000 to 50,000.

The polyisocyanate represented by the above R⁷—(NCO)ₕ₊₁ is not particularly limited as long as it has 2 or more isocyanate groups in a molecule. Examples include aliphatic diisocyanates, aromatic diisocyanates, alicyclic diisocyanates, biphenyl diisocyanates, and diisocyantes, triisocyanates, and tetraisocyantes of phenylmethane.

Examples of the aliphatic diisocyanate include a methylene diisocyanate, a dimethylene diisocyanate, a trimethylene diisocyanate, a tetramethylene diisocyanate, a pentamethylene diisocyanate, a hexamethylene diisocyanate, a dipropyl ether diisocyanate, a 2,2-dimethypentane diisocyanate, a 3-methoxyhexane diisocyanate, an octamethylene diisocyanate, a 2,2,4-trimethylpentane diisocyanate, a nonamethylene diisocyanate, a decamethylene diisocyanate, a 3-butoxyhexane diisocyanate, a 1,4-butylene glycol dipropyl ether diisocyanate, a thiodihexyl diisocyanate, a metaxylylene diisocyanate, a paraxylylene diisocyanate, and a tetramethylxylylene diisocyanate.

Examples of the aromatic diisocyanate include a methaphenilene diisocyanate, a paraphenilene diisocyanate, a 2,4-tolylene diisocyanate, a 2,6-tolylene diisocyanate, a dimethylbenzene diisocyanate, an ethylbenzene diisocyanate, an isopropylbenzene diisocyanate, a tolidine diisocyanate, a 1,4-naphthalene diisocyanate, a 1,5-naphthalene diisocyanate, a 2,6-naphthalene diisocyanate, and a 2,7-naphthalene diisocyanate.

Examples of the alicyclic diisocyanate include a hydrogenated xylylene diisocyanate and an isophorone diisocyanate.

Examples of the biphenyl diisocyanate include a biphenyl diisocyanate, a 3,3′-dimethylbiphenyl diisocyanate, and a 3,3′-dimethoxybiphenyl diisocyanate.

Examples of the diisocyanate of phenylmethane include a diphenylmethane-4,4′-diisocyanate, a 2,2′-dimethyldiphenylmethane-4,4′-diisocyanate, a diphenyldimethylmethane-4,4′-diisocyanate, a 2,5,2′,5′-tetramethyldiphenylmethane-4,4′-diisocyanate, a cyclohexylbis(4-isocyontophenyl)methane, a 3,3′-dimethoxydiphenylmethane-4,4′-diisocyanate, a 4,4′-dimethoxydiphenylmethane-3,3′-diisocyanate, a 4,4′-diethoxydiphenylmethane-3,3′-diisocyanate, a 2,2′-dimethyl-5,5′-dimethoxydiphenylmethane-4,4′-diisocyanate, a 3,3′-dichlorodiphenyldimethylmethane-4,4′-diisocyanate, and a benzophenone-3,3′-diisocyanate.

Examples of the triisocyanate of phenylmethane include a 1-methylbenzene-2,4,6-triisocyanate, a 1,3,5-trimethylbenzene-2,4,6-triisocyanate, a 1,3,7-naphthalene triisocyanate, a biphenyl-2,4,4′-triisocyanate, a diphenylmethane-2,4,4′-triisocyanate, a 3-methyldiphenylmethane-4,6,4′-triisocyanate, a triphenylmethane-4,4′,4″-triisocyanate, a 1,6,11-undecane triisocyanate, a 1,8-diisocyanate-4-isocyanate methyloctane, a 1,3,6-hexanemethylene triisocyanate, a bicycloheptane triisocyanate, and a tris(isocyanatephenyl)thiophosphate.

These polyisocyanate compounds can also be used in the form of dimers and trimers (isocyanurate bond) or reacted to amine and used as a biuret.

Further, polyisocyanates obtained by reacting these polyisocyanate compounds and polyols and having urethane bonds can also be used. For the polyols herein, 2 to 8 hydric polyols are preferable, with the polyols described earlier being preferable. When a 3 or more hydric polyisocyanate is used as the R⁷—(NCO)ₕ₊₁, the polyisocyanates having urethane bonds are preferable.

The polyether monoalcohol represented by the above formula HO—(R⁸—O)ₙ—R⁹ is not particularly limited as long as it is polyether of monohydric alcohol. Such a compound can be obtained by addition polymerization of an alkylene oxide, a styrene oxide or the like, such as an ethylene oxide, a propylene oxide, a butylene oxide, and an epichlorohydrin to monohydric alcohol.

The “monohydric alcohol” referred in the present description is represented by the following formula (3), (4), or (5).

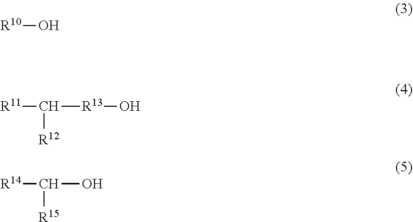

That is, R⁹ is a group in which the hydroxyl group is removed from the monohydric alcohol of the above formulae (3) to (5). In the above formulae (3) to (5), R¹⁰, R¹¹, R¹², R¹⁴ and R¹⁵ are hydrocarbon groups such as an alkyl group, an alkenyl group, an alkylaryl group, a cycloalkyl group, and a cycloalkenyl group.

Examples of the alkyl group include methyl, ethyl, propyl, isopropyl, butyl, isobutyl, tertiary butyl, pentyl, isopentyl, neopentyl, tertiary pentyl, hexyl, heptyl, octyl, 2-ethylhexyl, nonyl, decyl, undecyl, dodecyl, tridecyl, isotridecyl, myristyl, palmityl, stearyl, isostearyl, icosyl, docosyl, tetracosyl, triacontyl, 2-octyldodecyl, 2-dodecylhexadecyl, 2-tetradecyloctadecyl, and monomethyl branched-isostearyl.

Examples of the alkenyl group include vinyl, allyl, propenyl, isopropenyl, butenyl, pentenyl, isopentenyl, hexenyl, heptenyl, octenyl, nonenyl, decenyl, undecenyl, dodecenyl, tetradecenyl, and oleyl.

Examples of the alkylaryl group include phenyl, toluyl, xylyl, cumenyl, mesityl, benzyl, phenethyl, styryl, cinnamyl, benzhydryl, trityl, ethylphenyl, propylphenyl, butylphenyl, pentylphenyl, hexylphenyl, heptylphenyl, octylphenyl, nonylphenyl, α-naphthyl, and β-naphthyl.

Examples of the cycloalkyl group and the cycloalkenyl group include cyclopentyl, cyclohexyl, cycloheptyl, methyl cyclopentyl, methyl cyclohexyl, methyl cycloheptyl, cyclopentenyl, cyclohexenyl, cycloheptenyl, methylcyclopentenyl, methylcyclohexenyl, and methylcycloheptenyl.

In the above formula (4), Rⁿ is a hydrocarbon group, and examples include an alkylene group, an alkenylene group, an alkyl arylene group, a cycloalkylene group, and a cycloalkenylene group.

R⁹ is a hydrocarbon group, of which an alkyl group is preferable, and further the total number of carbon atoms thereof is preferably 8 to 36, and particularly preferably 12 to 24.

The alkylene oxide, styrene oxide or the like to be added can be homopolymerization or can be random polymerization or block polymerization of two or more thereof. The addition method can be a typical method. The polymerization degree n is 0 to 1000, preferably 1 to 200, and further preferably 10 to 200. The proportion of the ethylene group in R⁸ is preferably 50 to 100 wt. %, and further preferably 65 to 100 wt. %, of the entire R⁸.

The copolymer (hydrophobic modified polyether urethane) represented by the above formula (2) can be produced, for example, by heating at 80 to 90° C. for 1 to 3 hours and allowing to react as in the same manner as the general reaction of a polyether to an isocyanate.

When the polyether polyol (A) represented by R⁵—[(O—R⁶)ₖ—OH]ₘ, the polyisocyanate (B) represented by R⁷—(NCO)ₕ₊₁, and the polyether monoalcohol (C) represented by HO—(R⁸—O)ₙ—R⁹ are reacted, a byproduct can also be created other than the copolymer of the formula (2) structure. For example, when a diisocyanate is used, a C-B-A-B-C type copolymer represented by the formula (2) is produced as the main product, but other copolymers such as C-B-C type, C-B-(A-B)x-A-B-C type, and the like can be created as byproducts. In this instance, the formula (2) type copolymers are not particularly separated, and the copolymer in a state of mixture including the formula (2) type copolymers can be used for the present invention.

Particularly preferable examples include “(PEG-240/decyltetradeceth-20/HDI) copolymer (INCI name: PEG-240/HDI COPOLYMER BISDECYLTETRADECETH-20 ETHER)”, which is hydrophobic modified polyether urethane. This copolymer is commercially available from ADEKA CORPORATION under the product name “ADEKANOL GT-700”.

The amount of (b) the hydrophobic modified polyether urethane incorporated in the composition of the present invention relative to the total amount of the cosmetic is 0.1 mass % or more, preferably 0.2 mass % or more, and more preferably 0.3 mass % or more. The upper limit of amount incorporated relative to the total amount of the cosmetic is 5 mass % or less, preferably 3 mass % or less, and more preferably 2 mass % or less. That is, the range of amount incorporated can be set, for example, from 0.1 to 5 mass %, 0.2 to 3 mass %, and 0.3 to 2 mass %, but is not limited thereto. When an amount of (b) the hydrophobic modified polyether urethane incorporated is less than 0.1 mass %, an effect of imparting firmness is not sufficiently exhibited, and the base stability also tends to be affected. When an amount incorporated is more than 5 mass %, stickiness can be caused.

### (c) Carboxyvinyl Polymer

The carboxyvinyl polymer (ingredient c) in the present invention is a water soluble polymer having polyacrylic acid as the main chain and a carboxy group. The carboxyvinyl polymer used in the present invention can be those called “Carbomer” by the cosmetic labeling name, or those called “carboxyvinyl polymer” by quasi-drug additive name.

Carboxyvinyl polymers (Carbomer) have been commonly used in cosmetics and the like as an aqueous thickener, and a commercial product can be preferably used. Examples include Synthalen L and Synthalen F (manufactured by FUJIFILM Wako Pure Chemical Corporation), Carbopol 981 (manufactured by Lubrizol Advanced Materials), HIVISWAKO (manufactured by FUJIFILM Wako Pure Chemical Corporation), AQUPEC (manufactured by Sumitomo Seika Chemicals Company, Limited), and NTC-CARBOMER (manufactured by Guangzhou Tinci silicone technology).

The carboxyvinyl polymer used in the present invention is not particularly limited and, for example, it is preferable to select those having a viscosity (room temperature) of a 0.5 mass % aqueous solution ranging from 1,000 to 80,000 mPa·s, and more preferably from 3,000 to 50,000 mPa·s.

The amount of (c) the carboxyvinyl polymer incorporated relative to the total amount of the cosmetic is 0.001 mass % or more, and preferably 0.01 mass % or more. The upper limit of amount incorporated relative to the total amount of the cosmetic is 1 mass % or less, and preferably 0.5 mass % or less. That is, the range of amount incorporated relative to the total amount of the cosmetic can be set from 0.001 to 1 mass %, or 0.01 to 0.5 mass %, but is not limited thereto. However, when an amount of (c) the carboxyvinyl polymer incorporated is less than 0.001 mass %, a stabilizing effect may not be obtained, whereas when more than 1 mass % is incorporated, the cosmetic has an increased viscosity and, in some cases, becomes difficult to use by discharging from a dispenser.

In the cosmetic of the present invention, the ratio of (b) the hydrophobic modified polyether urethane to (c) the carboxyvinyl polymer incorporated ((b)/(c): mass ratio) is preferably within a range from 9 to 20, and more preferably within a range from 10 to 15. When a ratio of incorporation ((b)/(c)) described above is less than 9, the cosmetic has an increased viscosity and, in some cases, becomes difficult to use by discharging from a dispenser. On the other hand, when a ratio of incorporation is more than 20, the emulsion stability at a high temperature tends to decrease.

The cosmetic of the present invention is an oil-in-water emulsion product and includes an oil content in the internal phase. The oil content is not particularly limited as long as it is an oily ingredient that can be typically incorporated in cosmetics, and examples include a silicone oil and a hydrocarbon oil.

Examples of the silicone oil include chain silicones such as dimethylpolysiloxane (dimethicone), methylphenylpolysiloxane, and methyl hydrogen polysiloxane; cyclic silicones such as octamethylcyclotetrasiloxane, decamethylcyclopentasiloxane, and dodecamethylcyclohexane. Commercial products such as silicone KF-96A-6T (manufactured by Shin-Etsu Chemical Co., Ltd.) are preferably used.

Examples of the hydrocarbon oil include a liquid paraffin, a squalane, a squalene, a paraffin, an isoparaffin, a ceresine, an olefin oligomer (hydrogenated polydecene), a hydrogenated polybutene.

The amount of the oil content incorporated in the cosmetic of the present invention is not particularly limited and is typically 0.05 to 30 mass %, preferably 0.1 to 20 mass %, and more preferably 0.5 to 10 mass %.

The oil-in-water emulsion cosmetic of the present invention is preferably prepared so that the internal phase (oil phase) forms an a gel as in Patent Documents 1 and 2. That is, the cosmetic of the present invention preferably includes both an amphiphilic substance and a surfactant, which are considered to be required for the formation of the a gel.

The amphiphilic substance is preferably a higher alcohol and/or a higher fatty acid having 16 or more carbon atoms. Specific examples includes higher fatty acids such as lauric acid, myristic acid, palmitic acid, stearic acid, behenic acid (behenyl acid), oleic acid, 12-hydroxystearic acid, undecylenic acid, tall acid, lanolin fatty acid, isostearic acid, linoleic acid, linolenic acid, and eicosapentaenoic acid, and linear or branched chain higher alcohols such as lauryl alcohol, cetyl alcohol, stearyl alcohol, behenyl alcohol, myristyl alcohol, oleyl alcohol, cetostearyl alcohol, monostearyl glyceryl ether (batyl alcohol), 2-decyltetradecinol, lanolin alcohol, cholesterol, hexyldodecanol, isostearyl alcohol, and octyldodecanol. Of these, higher alcohols having 16 to 30 carbon atoms are preferably used.

(a) The carboxy modified silicone is different from the a gel described in Patent Document 2 in the aspect that the oil phase (a gel) of the present invention included in the surfactant contributing to the a gel formation includes (a) the carboxy modified silicone as a surfactant. As a result, even when a thickener other than (b) the hydrophobic modified polyether urethane (that is, (c) the carboxyvinyl polymer) is added, a feel derived from (b) the hydrophobic modified polyether urethane when touched is not affected, but rather the cosmetic is easily spreadable and has a smooth usability.

Surfactants other than (a) the carboxy modified silicone can also be incorporated in the cosmetic of the present invention. For the “other surfactants” than the carboxy modified silicone, anionic, cationic or zwitterionic surfactants, or nonionic surfactants can be used, and are not limited thereto.

Specific examples of the “other surfactants” include, but are not limited thereto, fatty acid soaps formed from behenic acid and potassium hydroxide, fatty acid soaps formed from stearic acid and potassium hydroxide, and POE sorbitan fatty acid esters such as sodium cetyl sulfate, behenyl trimethyl ammonium chloride, stearyl trimethyl ammonium chloride, POE sorbitan monostearate, and POE sorbitan monooleate, POE glycerin fatty acid esters such as POE glycerin monoisostearate and PEG-5 glyceryl stearate, POE alkyl ethers such as POE stearyl ether and POE cholestanol ether, POE alkyl phenyl ethers such as POE nonyl phenyl ether, poloxamers such as Pluronic (registered trademark), POE-POP alkyl ethers such as POE-POP cetyl ether, poloxamines (tetra POE-tetra POP ethylenediamine condensate) such as Tetronic (registered trademark), POE castor oil hydrogenated castor oil derivatives such as POE castor oil and POE hydrogenated castor oil, a POE beeswax-lanolin derivative, alkanolamide, POE propylene glycol fatty acid ester, POE alkylamine, POE fatty acid amide, sucrose fatty acid ester, a POE nonyl phenyl formaldehyde condensate, an alkyl ethoxy dimethylamine oxide, and trioleyl phosphate. Of these, nonionic surfactants such as POE glycerin fatty acid ester are preferable. The amount of the “other surfactants” incorporated is typically 0.1 to 5 mass %, and preferably 0.5 to 3 mass %, relative to the total amount of the cosmetic.

Other optional ingredients typically used for cosmetics, quasi-drugs and the like can be incorporated in the cosmetic of the present invention in addition to the above ingredients within the range that does not affect the effects of the present invention. The other optional ingredients are not limited and examples include a moisturizer, a preservative, various drugs, a buffer, and a fragrance.

The cosmetic of the present invention is suitably provided in the form of having a high water content with a low oil content, particularly such as in the form of lotions, milky lotions, and serums. For example, it is preferable that the cosmetic of the present invention contain 50 mass % or more, preferably 60 mass % or more, and more preferably 70 mass % or more, of water relative to the total amount of the cosmetic.

### EXAMPLES

Hereinafter, the present invention will be further described in detail in reference to examples but is in no way limited to these descriptions. The amount incorporated means mass % unless otherwise specified.

The cosmetics having the formulations shown in the following Table 1 (Examples and Comparative Examples) were evaluated for “firmness feel”, “absence of stickiness”, “stability over time”, “viscosity”, and “dischargeability from a dispenser.”

The evaluation methods and evaluation criteria for each evaluation item are as follows.

### <Firmness Feel and Stickiness Feel>

Professional panelists (10 women) were asked to use the composition of each example and evaluated for the firmness feel and the stickiness feel based on the following evaluation criteria.

### (Evaluation Criteria)

A+: 9 or more panelists answered “sensed a firmness feel” or “no stickiness.”

A: 7 to 8 panelists answered “sensed a firmness feel” or “no stickiness.”

B: 4 to 6 panelists answered “sensed a firmness feel” or “no stickiness.”

C: 3 or less panelists answered “sensed a firmness feel” or “no stickiness.”

### <Stability Over Time>

The composition of each example was stored for 1 month at 0° C. and 50° C., and the visual observation result on a state of the composition at 50° C. was evaluated with 0° C. as the reference in accordance with the following criteria.

### (Evaluation Criteria)

A+: Same as the initial state even after time lapsed.

A: Viscosity slightly declined from the initial state after time lapsed.

B: Viscosity declined as time lapsed and separation tended to be found.

C: Not producible (Clouded like a milky lotion from the initial state and separated.)

### <Viscosity>

Viscosity measurement was carried out at 25° C. using a cone-plate viscoelasticity measuring system MCR302 (manufactured by Anton Paar Germany GmbH) and CP25-2 as a measuring jig.

### (Evaluation Criteria 1)

Viscosity (Pa·s) when a shear velocity of 0.001γ/s was applied

A: 500 Pa·s or more

B: 100 Pa·s or more and less than 500 Pa·s

C: Less than 100 Pa·s

### (Evaluation Criteria 2)

Viscosity (Pa·s) when a shear velocity of 10 y/s was applied

A: 5 Pa·s or more

B: 1 Pa·s or more and less than 5 Pa·s

C: Less than 1 Pa·s

<Dischargeability from a Dispenser>

Dischargeability of the compositions from a dispenser at a low temperature condition (−5° C.) was confirmed.

### (Evaluation Criteria)

A: Dischargeable from a dispenser by lightly pressing with a finger.

B: Dischargeable from a dispenser by strongly pressing with a finger.

C: Unable to press down a dispenser with a finger and undischargeable.

TABLE 1

| | | | | Comparative | Comparative | Comparative |
|---|---|---|---|---|---|---|
| | Example 1 | Example 2 | Example 3 | Example 1 | Example 2 | Example 3 |
| Ion exchanged water | Balance | Balance | Balance | Balance | Balance | Balance |
| Alcohol | 3 | 3 | 3 | 3 | 3 | 3 |
| Glycerin | 2 | 2 | 2 | 2 | 2 | 2 |
| Dipropylene glycol | 6 | 6 | 6 | 6 | 6 | 6 |
| 1,3-Butylene glycol | 6 | 6 | 6 | 6 | 6 | 6 |
| PEG-6 | 3 | 3 | 3 | 3 | 3 | 3 |
| (a) Carboxy modified | 0.6 | 0.6 | 0.6 | — | 0.6 | 0.6 |
| silicone (*1) | | | | | | |
| (b) Hydrophobic | 0.8 | 0.8 | 0.8 | 0.8 | — | 1 |
| modified polyether | | | | | | |
| urethane (*2) | | | | | | |
| (c) Carboxyvinyl | 0.06 | 0.04 | 0.07 | 0.04 | 0.1 | — |
| polymer | | | | | | |
| PEG-5 Glyceryl stearate | 1.28 | 1.28 | 1.28 | 1.28 | 1.28 | 1.28 |
| Behenyl alcohol | 0.8 | 0.8 | 0.8 | 0.8 | 0.8 | 0.8 |
| Dimethicone | 1.8 | 1.8 | 1.8 | 1.8 | 1.8 | 1.8 |
| Hydrogenated | 1.8 | 1.8 | 1.8 | 1.8 | 1.8 | 1.8 |
| polydecene | | | | | | |
| Triethanolamine | 0.3 | 0.3 | 0.3 | 0.2 | 0.3 | 0.2 |
| EDTA-2Na | 0.03 | 0.03 | 0.03 | 0.03 | 0.03 | 0.03 |
| Sodium metabisulfite | 0.003 | 0.003 | 0.003 | 0.003 | 0.003 | 0.003 |
| Phenoxyethanol | 0.5 | 0.5 | 0.5 | 0.5 | 0.5 | 0.5 |
| Total | 100 | 100 | 100 | 100 | 100 | 100 |
| (b)/(C) | 13.3 | 20 | 11.4 | 20 | 0 | — |
| Stability over time | A+ | A | A+ | C | B | B |
| Viscosity (Pa·S) [Shear | A | B | A | — | A | C |
| velocity 0.001 γ/s] | | | | | | |
| Viscosity (Pa·S) [Shear | A | B | A | — | C | A |
| velocity 10 γ/s] | | | | | | |
| Firmness feel | A+ | A | A | — | C | A |
| Absence of stickiness | A | A | A | — | B | C |
| Dischargeability from | A | A | B | — | A | B |
| dispenser | | | | | | |

(*1) Carboxydecyl trisiloxane
(*2) (PEG-240/decyltetradeceth-20/HDI) copolymer

As shown in Table 1, the cosmetics containing all of (a) the carboxy modified silicone, (b) the hydrophobic modified polyether urethane, and (c) the carboxyvinyl polymer had good results in the aspects of the usability (firmness feel and stickiness feel) and the stability over time (Examples 1 to 3). In contrast, Comparative Example 1 in which (a) the carboxy modified silicone was not incorporated lost the activator balance of HLB, caused poor emulsification, and separated immediately after produced. Comparative Example 2 in which (b) the hydrophobic modified polyether urethane was not incorporated failed to impart firmness to the skin and had deteriorated viscosities at the stability over time and the shear velocity of 10γ/s. Additionally, Comparative Example 3 in which (c) the carboxyvinyl polymer was not incorporated had deteriorated viscosities at the stability over time and the shear velocity of 0.001γ/s.

1. An oil-in-water emulsion cosmetic comprising:
(a) a carboxy modified silicone;
(b) hydrophobic modified polyether urethane; and
(c) a carboxyvinyl polymer.

2. The cosmetic according to claim 1, wherein said (a) carboxy modified silicone is a compound represented by the following formula (1): 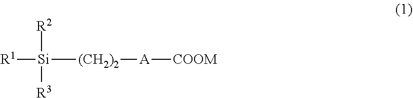
wherein, at least one of R¹ to R³ is a functional group represented by —O—Si(R⁴)₃, provided that R⁴ is an alkyl group having 1 to 6 carbon atoms or a phenyl group, and remining of R¹ to R³, which may be the same or different, are substituted or unsubstituted monohydric hydrocarbon groups;
M is a hydrogen atom, a metal atom, or an organic cation; A is a linear or branched chain alkylene group represented by C_{q}H_{2q}; and q is an integer of 0 to 20.

3. The cosmetic according to claim 1, wherein said (b) hydrophobic modified polyether urethane is a compound represented by the following formula (2):
R⁵—{(O—R⁶)ₖ—OCONH—R⁷[—NHCOO—(R⁸—O)ₙ—R⁹]ₕ)}ₘ (2)
Wherein, R⁵, R⁶ and R⁸ each independently represent a hydrocarbon group having 2 to 4 carbon atoms; R⁷ represents a hydrocarbon group having 1 to 10 carbon atoms and optionally having a urethane bond; R⁹ represents a hydrocarbon group having 8 to 36 carbon atoms; m is the number of 2 or more; h is the number of 1 or more; k is the number of 1 to 500; and n is the number of 1 to 200.

4. The cosmetic according to claim 1, wherein an amount of said (a) carboxy modified silicone relative to the total amount of the cosmetic is 0.1 to 5 mass %, an amount of said (b) hydrophobic modified polyether urethane relative to the total amount of the cosmetic is 0.1 to 5 mass %, and an amount of said (c) carboxyvinyl polymer relative to the total amount of the cosmetic is 0.001 to 1 mass %.

5. The cosmetic according to claim 1, wherein a ratio of the amount of said (b) hydrophobic modified polyether urethane to the (c) the amount of said (c) carboxyvinyl polymer, ((b)/(c)), is within a range from 9 to 20.
